(19) United States
(12) Patent Application Publication
(10) Pub. No.: US 2016/0345887 A1
(43) Pub. Date: Dec. 1, 2016

(54) MOISTURE FEELING EVALUATION DEVICE, MOISTURE FEELING EVALUATION METHOD, AND MOISTURE FEELING EVALUATION PROGRAM

(71) Applicant: FUJIFILM Corporation, Tokyo (JP)

(72) Inventors: Naoko YOSHIDA, Ashigara-kami-gun (JP); Ikuko OHGARU, Ashigara-kami-gun (JP); Eriko IKEDA, Ashigara-kami-gun (JP)

(73) Assignee: FUJIFILM Corporation, Tokyo (JP)

(21) Appl. No.: 15/237,135

(22) Filed: Aug. 15, 2016

Related U.S. Application Data
(63) Continuation of application No. PCT/JP2015/051802, filed on Jan. 23, 2015.

(30) Foreign Application Priority Data
Feb. 21, 2014 (JP) 2014-031460

(51) Int. Cl. A61B 5/00; A61B 5/107; A61B 5/103

(52) U.S. Cl. CPC A61B 5/443; A61B 5/0077; A61B 5/442; A61B 5/1032; A61B 5/1077; A61B 5/1072; A61B 5/1079; A61B 2576/02

(57) ABSTRACT

Disclosed is a moisture feeling evaluation device. In the moisture feeling evaluation device, an image input unit 1 receives an input of a captured image obtained by imaging a face F of a subject from the front thereof, a brightness-color index calculation unit 10 calculates values of brightness-color components including brightness, redness, and yellowness of skin from the captured image input to the image input unit 1 and calculates brightness-color indexes based on the values of the brightness-color components, a shape index calculation unit 11 detects an uneven portion of the skin from the captured image input to the image input unit 1 and calculates a shape index based on the amount of the uneven portion, and a moisture feeling evaluation unit 5 evaluates a feeling of visible moisture of the face F of the subject based on the brightness-color indexes and the shape index.

### CROSS-REFERENCE TO RELATED APPLICATIONS

This application is a continuation of PCT International Application No. PCT/JP2015/51802 filed on Jan. 23, 2015, which claims priority under 35 U.S.C. §119(a) to Japanese Patent Application No. 2014-031460 filed on Feb. 21, 2014. Each of the above applications is hereby expressly incorporated by reference, in its entirety, into the present application.

### BACKGROUND OF THE INVENTION

1. Field of the Invention

The present invention relates to a moisture feeling evaluation device, a moisture feeling evaluation method, and a moisture feeling evaluation program, and particularly to a moisture feeling evaluation device, a moisture feeling evaluation method, and a moisture feeling evaluation program that evaluate a feeling of moisture based on a captured image obtained by imaging the face of a subject.

2. Description of the Related Art

In recent years, in a cosmetic field, various methods of evaluating a feeling of moisture of the skin have been proposed. In general, the skin having the feeling of moisture refers to fresh and youthful skin that looks like it contains moisture. However, when the amount of moisture or the like of the skin is simply measured to evaluate the feeling of moisture, it is not possible to obtain a high correlation with functional evaluation that an observer actually views the skin for evaluation. Accordingly, an evaluation method having a high correlation with functional evaluation is demanded.

As such a moisture feeling evaluation method, for example, as disclosed in JP2003-024282A, a skin state evaluation method of evaluating a feeling of moisture of the skin by associating a mean fractional coefficient and/or a fluctuation coefficient of the mean fractional coefficient on a surface of the skin of a person and a functional index indicating the state of the skin has been proposed.

### SUMMARY OF THE INVENTION

However, in the evaluation method disclosed in JP2003-024282A, the feeling of moisture is evaluated based on partial physical characteristics of the skin, and thus, while it is possible to obtain a high correlation with respect to functional evaluation when partially observing the skin, it is not possible to obtain a high correlation with respect to functional evaluation for evaluating the feeling of moisture (feeling of visible moisture) when generally viewing the skin. In reality, when evaluating the feeling of moisture by observing the skin, the feeling of visible moisture is evaluated by generally viewing the skin. Thus, an evaluation result which is greatly different from a sensation of the feeling of moisture when actually viewing the skin is obtained. In particular, in the case of the face, the state of the skin is complicatedly changed according to positions, and thus, it is difficult to obtain an evaluation result having a high correlation with the sensation of the feeling of moisture when actually viewing the skin from evaluation for only a part of the skin or from only one type of physical characteristic of the skin.

In order to solve these problems, an object of the invention is to provide a moisture feeling evaluation device, a moisture feeling evaluation method, and a moisture feeling evaluation program capable of evaluating a feeling of visible moisture of the face with high accuracy.

According to an aspect of the invention, there is provided a moisture feeling evaluation device including: an image input unit that receives an input of a captured image obtained by imaging a face of a subject from the front thereof; a brightness-color index calculation unit that calculates a value of a brightness-color component including brightness, redness, and yellowness of skin from the captured image input to the image input unit and calculates a brightness-color index based on the value of the brightness-color component; a shape index calculation unit that detects an uneven portion of the skin from the captured image input to the image input unit and calculates a shape index based on the amount of the uneven portion; and a moisture feeling evaluation unit that evaluates a feeling of visible moisture of the face of the subject based on the brightness-color indexes and the shape index.

Here, it is preferable that the brightness-color index calculation unit includes a brightness calculation unit that calculates the brightness of the skin, a dullness calculation unit that calculates the amount of a dullness portion in the skin, a stain calculation unit that calculates the amount of a stain portion in the skin, and a color irregularity calculation unit that calculates the amount of a color irregularity portion in the skin, and the shape index calculation unit includes a wrinkle calculation unit that calculates the amount of a wrinkle portion in the skin, a pore calculation unit that calculates the amount of a pore portion in the skin, and a contour recess amount calculation unit that calculates the amount of a recess generated in a cheek contour shape ranging from an ear to the mouth.

Further, it is preferable that the brightness calculation unit sets a first evaluation region in the captured image, and calculates an average value of brightnesses in the first evaluation region as the brightness of the skin, the dullness calculation unit sets a second evaluation region in the captured image, detects the dullness portion from the second evaluation region based on a brightness value, a redness value, and a yellowness value, and calculates a total area of the dullness portion, an area ratio of the dullness portion with respect to the second evaluation region, or a shade of the dullness portion, as the amount of the dullness portion, the stain calculation unit sets a third evaluation region in the captured image, and detects the stain portion where the brightness component value or the color component value is locally changed from the third evaluation region based on its size, and calculates a total area of the stain portion, an area ratio of the stain portion with respect to the third evaluation region, a shade of the stain portion, or the number of the stain portions in the third evaluation region, as the amount of the stain portion, and the color irregularity calculation unit sets a fourth evaluation region in the captured image, and detects the color irregularity portion where the brightness component value or the color component value is locally changed and its size is larger than that of the stain portion from the fourth evaluation region, and calculates a total area of the color irregularity portion, an area ratio of the color irregularity portion with respect to the fourth evaluation region, a shade of the color irregularity portion, or the number of the color irregularity portions in the fourth evaluation region, as the amount of the color irregularity portion.

Further, it is preferable that the wrinkle calculation unit sets a fifth evaluation region that extends from the nostrils to the corners of the mouth in the captured image, detects the wrinkle portion where the brightness decreases in the fifth evaluation region, and calculates a total area of the wrinkle portion, an area ratio of the wrinkle portion with respect to the fifth evaluation region, a shade of the wrinkle portion, or the lengths of the wrinkle portion, as the amount of the wrinkle portion, the pore calculation unit sets a sixth evaluation region in the captured image, detects the pore portion where the brightness component value or the color component value is locally changed and its size is smaller than that of the stain portion from the sixth evaluation region, and calculates a total area of the pore portion, an area ratio of the pore portion with respect to the sixth evaluation region, a shade of the pore portion, or the number of the pore portions in the sixth evaluation region, as the amount of the pore portion, and the contour recess amount calculation unit detects the cheek contour shape in the captured image, draws a straight line in a downward direction from an outermost portion in the cheek contour shape, and calculates a distance from the straight line to the cheek contour shape as the recess amount.

Further, the moisture feeling evaluation unit may evaluate the feeling of visible moisture based on a linear sum of the brightness-color indexes and the shape index with respect to a reference value (visual evaluation value) of the feeling of visible moisture which is obtained in advance by visually evaluating faces of plural subjects having different brightness-color component values and different amounts of uneven portions.

Further, it is preferable that the moisture feeling evaluation device further includes a database that stores each coefficient for calculating the linear sum of the brightness-color indexes and the shape index with respect to the reference value, and the moisture feeling evaluation unit calculates an evaluation value of the feeling of visible moisture with reference to the database based on the brightness-color index values calculated in the brightness-color index calculation unit and the shape index value calculated in the shape index calculation unit.

According to another aspect of the invention, there is provided a moisture feeling evaluation method including: receiving an input of a captured image obtained by imaging a face of a subject; calculating a value of a brightness-color component including brightness, redness, and yellowness of skin from the captured image, and calculating a brightness-color index based on the value of the brightness-color component; detecting an uneven portion of the skin from the captured image, and calculating a shape index based on the amount of the uneven portion; and evaluating a feeling of visible moisture of the face of the subject based on the brightness-color indexes and the shape index.

According to still another aspect of the invention, there is provided a non-transitory computer-readable medium storing moisture feeling evaluation program that causes a computer to execute: a step of receiving an input of a captured image obtained by imaging a face of a subject; a step of calculating a value of a brightness-color component including brightness, redness, and yellowness of skin from the captured image, and calculating a brightness-color index based on the value of the brightness-color component; a step of detecting an uneven portion of the skin from the captured image, and calculating a shape index based on the amount of the uneven portion; and a step of evaluating a feeling of visible moisture of the face of the subject based on the brightness-color indexes and the shape index.

According to the invention, since a feeling of visible moisture of the face of a subject is evaluated based on a brightness-color index calculated based on brightness-color component values and a shape index calculated based on the amount of uneven portions, it is possible to evaluate the feeling of visible moisture of the face with high accuracy.

### BRIEF DESCRIPTION OF THE DRAWINGS

FIG. 1 is a block diagram illustrating a configuration of a moisture feeling evaluation device according to the invention.

FIG. 2 is a block diagram illustrating a configuration of a brightness-color index calculation unit.

FIG. 3 is a block diagram illustrating a configuration of a shape index calculation unit.

FIG. 4 is a diagram illustrating an evaluation region set in a face of a subject in a brightness calculation unit.

FIG. 5 is a diagram illustrating an evaluation region set in a face of a subject in a dullness calculation unit.

FIG. 6 is a diagram illustrating an evaluation region set in a face of a subject in a wrinkle calculation unit.

FIGS. 7A and 7B are diagrams illustrating a state where a recess amount of a cheek contour shape of a subject is calculated in a contour recess amount calculation unit.

FIG. 8 is a diagram illustrating the cheek contour shape of the subject detected in the contour recess amount calculation unit.

FIG. 9 is a diagram illustrating a method of detecting a dullness portion using linear discriminant analysis.

FIG. 10 is a diagram illustrating another method of detecting a dullness portion using linear discriminant analysis.

FIG. 11 is a diagram illustrating a correlation between a total index value for evaluation of a feeling of visible moisture and a functional evaluation value.

### DESCRIPTION OF THE PREFERRED EMBODIMENTS

Hereinafter, embodiments of the invention will be described with reference to the accompanying drawings.

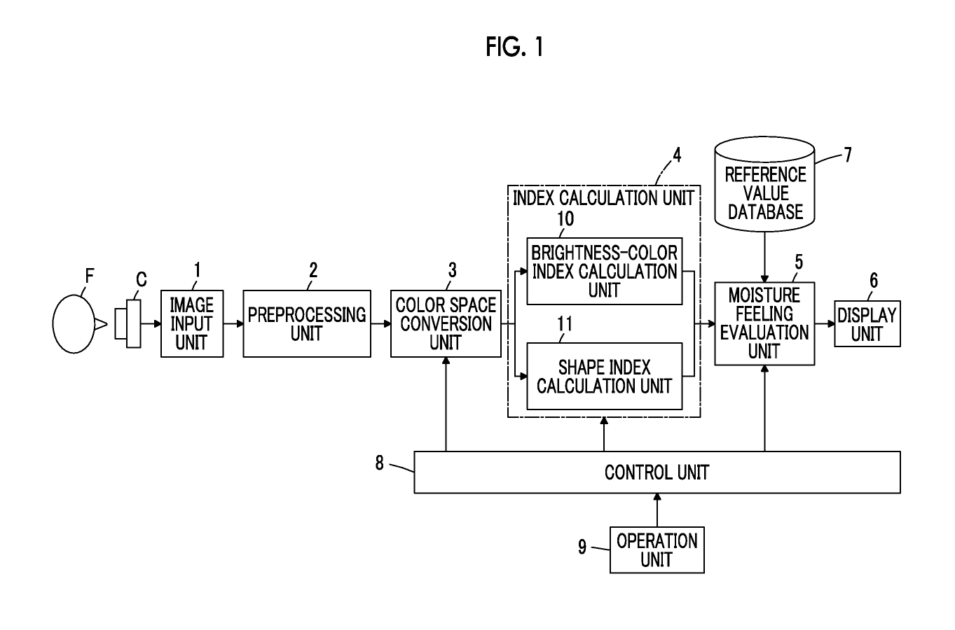

FIG. 1 shows a configuration of a moisture feeling evaluation device according to an embodiment of the invention. The moisture feeling evaluation device evaluates a feeling of visible moisture of a face F of a subject using a captured image obtained by imaging the face F of the subject from the front thereof using a camera C, and includes an image input unit 1 connected to the camera C. A preprocessing unit 2, a color space conversion unit 3, an index calculation unit 4, a moisture feeling evaluation unit 5 and a display unit 6 are sequentially connected to the image input unit 1. Further, a reference value database 7 is connected to the moisture feeling evaluation unit 5. In addition, a control unit 8 is connected to the color space conversion unit 3, the index calculation unit 4, and the moisture feeling evaluation unit 5, and an operation unit 9 is connected to the control unit 8.

The image input unit 1 receives an input of a captured image from the camera C that images the face F of the subject.

Here, it is preferable that the captured image is an image obtained by imaging the face F of the subject from the front thereof so that both ears of the face F of the subject are included. Further, it is assumed that the captured image input from the camera C has an RGB color space. As the camera C, any camera capable of imaging the face F of the subject may be used, and for example, a digital camera, a CCD camera, or the like may be used. Further, a captured image obtained by imaging using a mobile phone such as a smart phone may be used.

The preprocessing unit 2 performs preprocessing such as light intensity correction and noise removal with respect to a captured image input through the image input unit 1.

The color space conversion unit 3 converts a color space of a captured image input from the preprocessing unit 2 to generate a color space converted image. As the color space converted image, an image of which the color space is converted into an L*a*b* color space, an LCH color space, an YCC color space, or the like may be used, for example. In a case where the color space is converted into the L*a*b* color space, a D65 light source may be used as a calculation light source. Further, the color space conversion unit 3 generates a brightness component image and a color component image by dividing the generated color space converted image into a brightness component (luminance component) and a color component, respectively. Specifically, in the case of a color space converted image having the L*a*b* color space, the brightness component corresponds to an L* component, the color component corresponds to an a* component (complementary component corresponding to red and green), a b* component (complementary component corresponding to yellow and blue), a C* component (chroma component), a Hue component (hue component), and the like.

The index calculation unit 4 includes a brightness-color index calculation unit 10 and a shape index calculation unit 11 which are respectively connected to the color space conversion unit 3.

The brightness-color index calculation unit 10 receives inputs of the brightness component image and the color component image from the color space conversion unit 3, respectively, calculates values of brightness-color components including the brightness, redness, and yellowness of the skin from the brightness component image and the color component image, and calculates brightness-color indexes for evaluation of a feeling of visible moisture based on the values of the brightness-color components.

The shape index calculation unit 11 receives inputs of the bright component image, the color component image, and the captured image input to the image input unit 1 from the color space conversion unit 3, respectively, detects uneven portions of the skin from the brightness component image, the color component image, and the captured image, and calculates a shape index for evaluation of the feeling of visible moisture based on the amount of the uneven portions.

The brightness-color index calculation unit 10 and the shape index calculation unit 11 output the respectively calculated brightness-color indexes and shape index to the moisture feeling evaluation unit 5.

The reference value database 7 stores a relationship between the brightness-color index values, the shape index value, and a reference value of the feeling of visible moisture which is obtained in advance by visually evaluating faces of plural subjects having different brightness-color component values and different amounts of uneven portions. For example, a linear sum of the brightness-color index values and the shape index value may be calculated, and a function indicating a reference value of the feeling of visible moisture with respect to the linear sum may be stored.

The moisture feeling evaluation unit 5 evaluates a feeling of visible moisture of a face of a subject based on the brightness-color indexes and the shape index.

Specifically, on the basis of the brightness-color index values calculated in the brightness-color index calculation unit 10 and the shape index value calculated in the shape index calculation unit 11, an evaluation value of the feeling of visible moisture is calculated with reference to the reference value database 7. For example, the moisture feeling evaluation unit 5 may calculate the evaluation value of the feeling of visible moisture on the basis of the function indicating the reference value of the feeling of visible moisture with respect to the linear sum of the brightness-color index values and the shape index value stored in the reference value database 7.

Here, the feeling of moisture shows a fresh and youthful skin which is tightened and transparent and contains moisture, and the moisture feeling evaluation unit 5 evaluates the feeling of visible moisture indicating a feeling of moisture with respect to the entire face F of the subject.

The display unit 6 includes a display device such as an LCD, and displays an evaluation result of the feeling of visible moisture evaluated by the moisture feeling evaluation unit 5.

The operation unit 9 is a unit through which an operator performs an information input operation, and may be formed by a keyboard, a mouse, a track ball, a touch panel, or the like.

The control unit 8 controls the respective units in the moisture feeling evaluation device based on various command signals or the like input through the operation unit 9 by the operator.

The color space conversion unit 3, the index calculation unit 4, the moisture feeling evaluation unit 5, and the control unit 8 are configured by a CPU and an operation program that causes the CPU to perform various processes, but may be configured by a digital circuit. Further, a memory may be connected to the CPU through a signal line such as a bus, and for example, the captured image input to the image input unit 1, the brightness component image and the color component image generated in the color space conversion unit 3, the image generated in the index calculation unit 4, the evaluation result of the feeling of visible moisture calculated in the moisture feeling evaluation unit 5, and the like may be respectively stored in the memory. Further, the images stored in the memory and the evaluation result of the feeling of visible moisture may be displayed on the display unit 6 under the control of the control unit 8.

Next, the brightness-color index calculation unit 10 of the index calculation unit 4 will be described in detail.

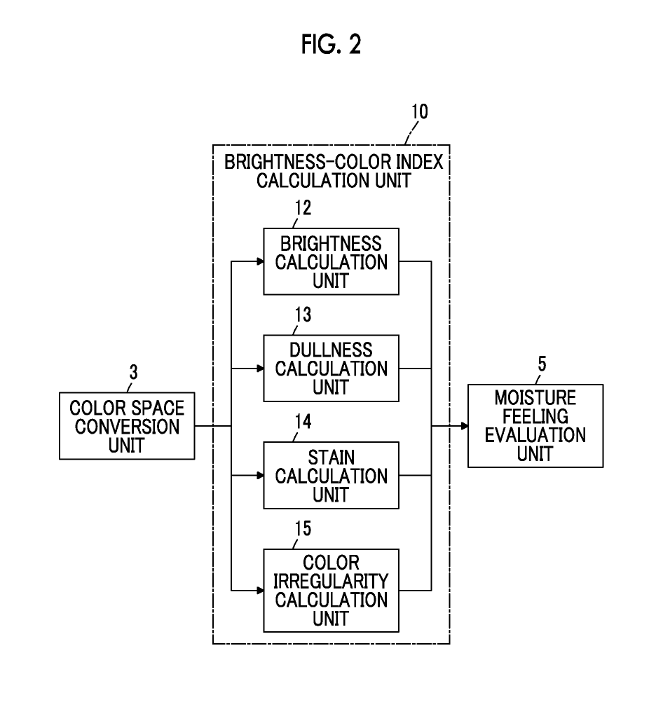

As shown in FIG. 2, the brightness-color index calculation unit 10 includes a brightness calculation unit 12, a dullness calculation unit 13, and a stain calculation unit 14, and a color irregularity calculation unit 15 which are respectively connected to the color space conversion unit 3 and the moisture feeling evaluation unit 5.

The brightness calculation unit 12 calculates the brightness of the skin as a brightness-color index. Specifically, the brightness calculation unit 12 sets an evaluation region R1 with respect to the brightness component image generated in the color space conversion unit 3. The evaluation region R1 may be set with respect to the entire face F or a cheek portion of the subject, for example. Subsequently, the brightness calculation unit 12 calculates an average value of brightness components in the evaluation region R1, as the brightness-color index, for example.

The dullness calculation unit 13 calculates the amount of dullness portions in the skin as the brightness-color index. Here, the dullness portion refers to a portion which occurs in the entire face, around the eyes, on the cheek, or the like, which is specifically in a state where redness of the skin decreases, yellowness thereof increases, gloss or transparency of the skin is reduced, and brightness thereof is reduced according to shading or the like due to unevenness of the surface of skin, so that the skin looks dark and its boundary becomes unclear.

Specifically, the dullness calculation unit 13 sets an evaluation region R2 with respect to the brightness component image and the color component image generated in the color space conversion unit 3. It is preferable that the evaluation region R2 is set around the eyes where tea dullness and yellow dullness are easily generated, and around the mouth where yellow dullness is easily generated. Subsequently, the dullness calculation unit 13 detects dullness portions from the evaluation region R2 based on a brightness value, a redness value, and a yellowness value, and for example, calculates a total area of the dullness portions in the evaluation region R2, an area ratio of the dullness portions with respect to the evaluation region R2, or a shade of the dullness portion based on the intensity of brightness, as the brightness-color index.

The stain calculation unit 14 calculates the amount of stain portions in the skin as the brightness-color index. Here, the stain portion refers to a portion where the brightness component value or the color component value is locally changed, which is a portion where the size (maximum width or diameter) is larger than 2 mm and is smaller than 50 mm, for example.

Specifically, the stain calculation unit 14 sets an evaluation region R3 with respect to the brightness component image and the color component image generated in the color space conversion unit 3. The evaluation region R3 may be set with respect to the entire face F or a cheek portion of the subject, for example. Subsequently, the stain calculation unit 14 detects stain portions where the brightness component value or the color component value is locally changed from the evaluation region R3 based on their sizes, and for example, calculates a total area of the stain portions in the evaluation region R3, an area ratio of the stain portions with respect to the evaluation region R3, a shade of the stain portion based on the intensity of brightness, or the number of the stain portions in the evaluation region R3, as the brightness-color index.

The color irregularity calculation unit 15 calculates the amount of color irregularity portions in the skin as the brightness-color index. Here, the color irregularity portion refers to a portion where the brightness component value or the color component value is locally changed, its size is larger than that of the stain portion, and its boundary is unclearly distributed.

Specifically, the color irregularity calculation unit 15 sets an evaluation region R4 with respect to the brightness component image and the color component image generated in the color space conversion unit 3. The evaluation region R4 may be set with respect to the entire face F or a cheek portion of the subject, for example. Subsequently, the color irregularity calculation unit 15 detects a color irregularity portion where the brightness component value or the color component value is locally changed and its size is larger than that of the stain portion from the evaluation region R4, and for example, calculates a total area of the color irregularity portions in the evaluation region R4, an area ratio of the color irregularity portions with respect to the evaluation region R4, a shade of the color irregularity portion based on the intensity of brightness, or the number of the color irregularity portions in the evaluation region R4, as the brightness-color index.

Then, the shape index calculation unit 11 of the index calculation unit 4 will be described in detail.

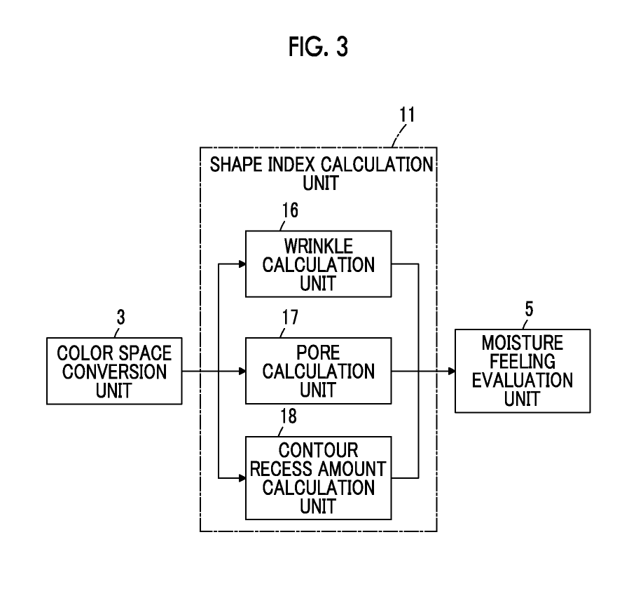

As shown in FIG. 3, the shape index calculation unit 11 includes a wrinkle calculation unit 16, a pore calculation unit 17, and a contour recess amount calculation unit 18 which are respectively connected to the color space conversion unit 3 and the moisture feeling evaluation unit 5.

The wrinkle calculation unit 16 calculates the amount of wrinkle portions in the skin as the shape index. Here, the wrinkle portion refers to a portion where the brightness component value or the color component value is locally changed and a shape which elongatedly extends in a predetermined direction is formed.

Specifically, the wrinkle calculation unit 16 sets an evaluation region R5 that extends from the nostrils to the corners of the mouth with respect to the brightness component image generated in the color space conversion unit 3. Subsequently, the wrinkle calculation unit 16 detects wrinkle portions where the brightness decreases in the evaluation region R5, and for example, calculates a total area of the wrinkle portions in the evaluation region R5, an area ratio of the wrinkle portions with respect to the evaluation region R5, a shade of the wrinkle portion based on the intensity of brightness, or the lengths of the wrinkle portions in the evaluation region R5, as the shape index.

The pore calculation unit 17 calculates the amount of pore portions in the skin as the shape index. Here, the pore portion refers to a portion where the brightness component value or the color component value is locally changed and its size is smaller than that of the stain portion.

Specifically, the pore calculation unit 17 sets an evaluation region R6 with respect to the brightness component image and the color component image generated in the color space conversion unit 3. The evaluation region R6 may be set with respect to the entire face F or a cheek portion of the subject, for example. Subsequently, the pore calculation unit 17 detects pore portions where the brightness component value or the color component value is locally changed and its size is smaller than that of the stain portion from the evaluation region R6, and for example, calculates a total area of the pore portions in the evaluation region R6, an area ratio of the pore portions with respect to the evaluation region R6, a shade of the pore portion based on the intensity of brightness, or the number of the pore portions in the evaluation region R6, as the shape index.

The contour recess amount calculation unit 18 detects a cheek contour shape ranging from an ear to the mouth in the face of a subject in a captured image input to the image input unit 1, and calculates the amount of recesses generated in the cheek contour shape as the shape index.

Next, an operation of this embodiment will be described.

First, a captured image obtained by imaging the face F of a subject using the camera C is input to the preprocessing unit 2 through the image input unit 1 of the moisture feeling evaluation device from the camera C, as shown in FIG. 1. The captured image is subject to preprocessing such as light source correction or noise removal, and is output to the color space conversion unit 3 from the preprocessing unit 2. Then, a color space of the captured image is converted into the L*a*b* color space by the color space conversion unit 3, for example, so that a color space converted image is generated. Further, the color space conversion unit 3 extracts a brightness component and a color component from the color space converted image, and generates a brightness component image and a color component image, respectively. For example, the color space conversion unit 3 may generate an L* component image as the brightness component image, and a C* component image, an a* component image, and a b* component image as the color component image.

The color space conversion unit 3 outputs the generated brightness component image and color component image to the brightness calculation unit 12, the dullness calculation unit 13, the stain calculation unit 14, and the color irregularity calculation unit 15 of the brightness-color index calculation unit 10, respectively.

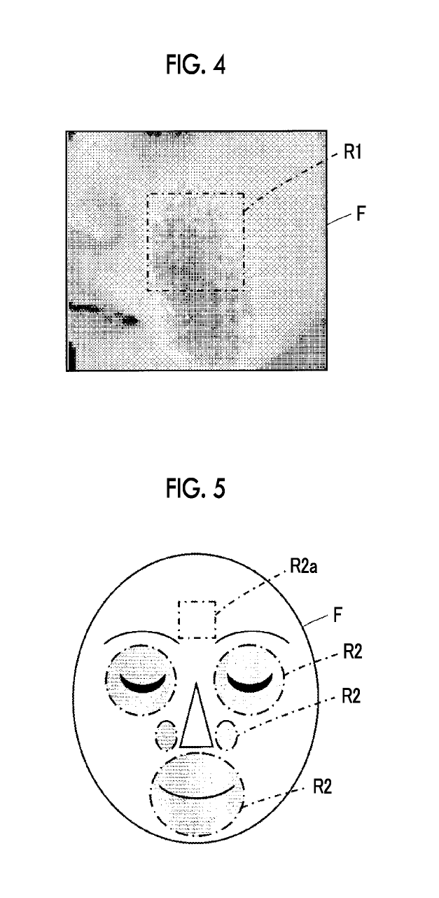

The brightness calculation unit 12 sets the evaluation region R1 in a cheek portion of the face F of the subject with respect to the L* component image input from the color space conversion unit 3, as shown in FIG. 4. Subsequently, the brightness calculation unit 12 calculates an average value of intensities of the L* components in the evaluation region R1 set with respect to the L* component image.

In general, it is known that young people's skin is white and bright, but the skin becomes yellow and dark and the feeling of visible moisture is generally reduced due to aging. Thus, it may be considered that the value of the L* component in the evaluation region R1 calculated in the brightness calculation unit 12 becomes an index indicating a change in the feeling of visible moisture due to aging. Specifically, as the value of the L* component becomes higher (becomes brighter), the feeling of visible moisture of the face F of the subject becomes higher. Accordingly, the average value of the L* components in the evaluation region R1 is output to the moisture feeling evaluation unit 5 from the brightness calculation unit 12, as the brightness-color index.

The dullness calculation unit 13 sets the evaluation region R2 in the face F of the subject with respect to the L* component image, the a* component image and the b* component image input from the color space conversion unit 3, and detects dullness portions from the set evaluation region R2.

Specifically, the dullness calculation unit 13 sets the evaluation region R2 in a region where dullness portions are easily generated in the face F of the subject, and sets a reference region R2a in a forehead region where dullness portions are not easily generated. As shown in FIG. 5, the evaluation region R2 may be set around the eyes where tea dullness and yellow dullness are easily generated, and around the nostrils where tea dullness is easily generated, and around the mouth where yellow dullness is easily generated. Further, the reference region R2a may be set in a range of about 1 cm² above the brow.

The dullness calculation unit 13 calculates an average value of the L* components, an average value of the a* components, and an average value of the b* components in the reference region R2a. Further, the dullness calculation unit 13 creates an ΔL* component image by subtracting the average value of the L* components in the reference region R2a from the L* component value in the evaluation region R2, creates an Δa* component image by subtracting the average value of the a* components in the reference region R2a from the a* component value in the evaluation region R2, and creates an Δb* component image by subtracting the average value of the b* components in the reference region R2a from the b* component value in the evaluation region R2.

Subsequently, the dullness calculation unit 13 detects dullness portions from the evaluation region R2 of the ΔL* component image, the Δa* component image, and the Δb* component image based on a predetermined threshold value which is set in advance. For example, when detecting tea dullness is detected, the dullness calculation unit 13 may detect a portion where the ΔL* component value is smaller than −5 and the Δa* component value exceeds 2.5 as the dullness portion. In the case of detecting yellow dullness, the dullness calculation unit 13 may detect a portion where the ΔL* component value is smaller than −5 and the Δb* component value exceeds 2.5 as the dullness portion.

Further, the dullness calculation unit 13 calculates a total area of the dullness portions detected in the evaluation region R2 as the brightness-color index, for example. Here, the dullness portion refers to a portion where the face F of the subject generally shows a dark impression, in which the feeling of visible moisture of the face F of the subject becomes higher as the total area becomes smaller.

The dullness calculation unit 13 outputs the calculated brightness-color index to the moisture feeling evaluation unit 5.

The stain calculation unit 14 sets the evaluation region R3 with respect to the brightness component image or the color component image input from the color space conversion unit 3, and detects stain portions from the evaluation region R3. For example, the stain calculation unit 14 may set the evaluation region R3 in a cheek portion of the face F of the subject with respect to the L* component image.

Here, the stain portions may be detected by generating a difference-of-Gaussian (DoG) image, for example. Specifically, DoG images having different Gaussian sizes are generated from an L* component image. Generally, a stain has a size of 2 mm to 10 mm, and a frequency of 0.05 cycle/mm to 0.25 cycle/mm. The stain calculation unit 14 performs DoG image processing so that a component having a stain frequency band is extracted. Further, when performing the DoG image processing, the stain calculation unit 14 may calculate a shape of each component from a binary image which is subject to threshold value processing, and may detect a component having a round shape and a circularity (4π×area)/circumference² of 2 mm to 10 mm as the stain portions.

Further, the stain portions may be detected by extracting a component of which a redness value and a yellowness value are smaller than a predetermined threshold value set in a dullness portion after performing the above-mentioned DoG image processing.

The stain calculation unit 14 may generate a DoG image using color component images such as an a* component image and a b* component image, in addition to the L* component image, to thereby detect the stain portions in a similar way to the above-described method. Further, the stain calculation unit 14 may generate a DoG image using a B channel in the RGB color space to detect the stain portions.

In addition, the stain calculation unit 14 may not generate a DoG image, and for example, may extract a component having a strength which is equal to or smaller than a predetermined threshold value from the L* component image, and may perform main component analysis and independent component analysis with respect to the extracted component, for example, to detect the stain portions.

Further, the stain calculation unit 14 calculates a total area of stain portions detected in the evaluation region R3 as the brightness-color index, for example. Here, the stain portion refers to a portion where the face F of the subject generally shows a dark impression and the feeling of visible moisture of the face F of the subject becomes higher as the total area becomes smaller.

The stain calculation unit 14 outputs the calculated brightness-color index to the moisture feeling evaluation unit 5.

The color irregularity calculation unit 15 sets the evaluation region R4 with respect to the brightness component image or the color component image input from the color space conversion unit 3, and detects color irregularity portions from the evaluation region R4. For example, the color irregularity calculation unit 15 may set the evaluation region R4 in a cheek portion of the face F of the subject with respect to the L* component image.

Here, the color irregularity portion may be detected by generating a DoG image, in a similar way to the detection of the stain portion. That is, DoG images having different Gaussian sizes are generated from an L* component image. Generally, a color irregularity has a size of about 10 mm or greater, and a frequency of 0.05 cycle/mm or greater. The color irregularity calculation unit 15 performs DoG image processing so that a component having a color irregularity frequency band is extracted. Further, when performing the DoG image processing, the color irregularity calculation unit 15 may calculate a shape of each component from a binary image which is subject to threshold value processing, and may detect a component having a round shape and a circularity (4π×area)/circumference² of about 10 mm or greater.

Further, the color irregularity portion may be detected by extracting a component of which a redness value and a yellowness value are smaller than a predetermined threshold value set in a dullness portion after performing the above-mentioned DoG image processing.

The color irregularity calculation unit 15 may generate a DoG image using color component images such as an a* component image or a b* component image, in addition to the L* component image, to thereby detect color irregularity portions in a similar way to the above-described method. Further, the color irregularity calculation unit 15 may generate a DoG image using a B channel in the RGB color space to detect the color irregularity portions.

In addition, the color irregularity calculation unit 15 calculates a total area of color irregularity portions detected in the evaluation region R4 as the brightness-color index, for example. Here, the color irregularity portion refers to a portion where the face F of the subject generally shows a dark impression, in which the feeling of visible moisture of the face F of the subject becomes higher as the total area becomes smaller.

The color irregularity calculation unit 15 outputs the calculated brightness-color index to the moisture feeling evaluation unit 5.

Further, the color space conversion unit 3 outputs the generated brightness component image and color component image to the wrinkle calculation unit 16 and the pore calculation unit 17 of the shape index calculation unit 11, and outputs a captured image input to the image input unit 1 to the contour recess amount calculation unit 18.

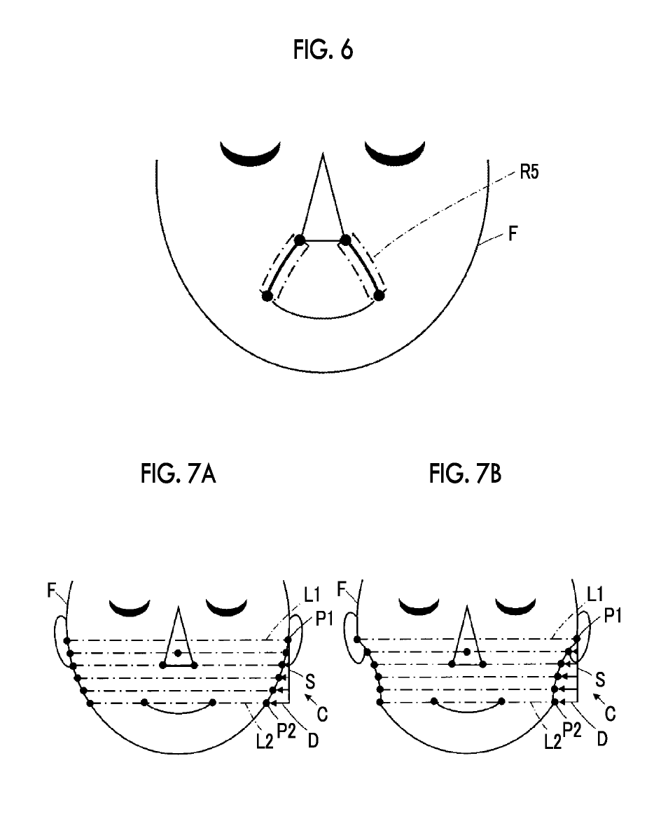

The wrinkle calculation unit 16 sets the evaluation region R5 that extends from the nostrils to the corners of the mouth with respect to the brightness component image or the color component image input from the color space conversion unit 3, as shown in FIG. 6, and detects wrinkle portions from the evaluation region R5. Further, the wrinkle calculation unit 16 sets a reference region R5a around the evaluation region R5, and calculates an average value of L* components in the reference region R5a.

Subsequently, the wrinkle calculation unit 16 creates an ΔL* component image obtained by subtracting the average value of the L* components in the reference region R5a from the L* component value in the evaluation region R5, and detects wrinkle portions from the evaluation region R5 of the ΔL* component image based on a predetermined threshold value which is set in advance. For example, a portion where the ΔL* component value is smaller than 10 in the evaluation region R5 may be detected as the wrinkle portion.

Further, the wrinkle calculation unit 16 calculates a total area of wrinkle portions detected in the evaluation region R5 as the shape index, for example. Here, the wrinkle portion detected in the evaluation region R5 refers to a portion which is a so-called nasolabial fold, in which the face F of the subject generally shows an impression as an uneven portion where shadow is generated and the feeling of visible moisture of the face F of the subject becomes higher as the total area becomes smaller.

The wrinkle calculation unit 16 outputs the calculated shape index to the moisture feeling evaluation unit 5.

The pore calculation unit 17 sets the evaluation region R6 with respect to the brightness component image or the color component image input from the color space conversion unit 3, and detects pore portions from the evaluation region R6. For example, the pore calculation unit 17 may set the evaluation region R6 in a cheek portion of the face F of the subject with respect to the L* component image.

Here, the pore portions may be detected by generating a DoG image, in a similar way to the case where the stain portions are detected. That is, DoG images having different Gaussian sizes are generated from an L* component image. Generally, a pore has a size of 0.5 mm to 2 mm, and a frequency of 0.25 cycle/mm to 1.0 cycle/mm. The pore calculation unit 17 performs DoG image processing so that a component having a pore frequency band is extracted. Further, when performing the DoG image processing, the pore calculation unit 17 may calculate a shape of each component from a binary image which is subject to threshold value processing, and may detect a component having a round shape and a circularity (4π×area)/circumference² of 0.5 mm to 2 mm as the pore portions.

Further, the pore portions may be detected by extracting a component of which a redness value and a yellowness value are smaller than a predetermined threshold value set in a dullness portion after performing the above-mentioned DoG image processing.

The pore calculation unit 17 may generate a DoG image using, color component images such as an a* component image and a b* component image, in addition to the L* component image, to thereby detect the pore portions in a similar way to the above-described method. Further, the pore calculation unit 17 may generate a DoG image using a B channel in the RGB color space to detect the pore portions.

In addition, the pore calculation unit 17 calculates a total area of pore portions detected in the evaluation region R6 as the shape index, for example. Here, the pore portion refers to a portion where the face F of the subject generally shows an impression which is an uneven portion which generates a shadow, in which the feeling of visible moisture of the face F of the subject becomes higher as the total area becomes smaller.

The pore calculation unit 17 outputs the calculated shape index to the moisture feeling evaluation unit 5.

The contour recess amount calculation unit 18 detects a contour shape of a cheek from an ear to the mouth in the captured image input from the color space conversion unit 3, draws a straight line in a downward direction from an outermost portion in the cheek contour shape, and calculates a distance from the straight line to the cheek contour shape as the recess amount.

Specifically, the contour recess amount calculation unit 18 detects a contour of the face F of the subject in the captured image, and as shown in FIGS. 7A and 7B, draws plural horizontal lines from a horizontal line L1 passing through the center of the ears to a horizontal line L2 passing through the mouth at uniform intervals, and detects an intersection where each horizontal line intersects the contour. Thus, it is possible to detect a contour shape from an intersection P1 with respect to the horizontal line L1 to an intersection P2 with respect to the horizontal line L2, that is, a contour shape C of the cheek from the ear to the mouth.

Here, FIG. 7A shows a contour shape C of a cheek of a subject having a high feeling of visible moisture, and FIG. 7B shows a contour shape C of a cheek of a subject having a low feeling of visible moisture. With respect to the contour shape C of the cheek of the subject having the high feeling of visible moisture, the contour shape C of the cheek of the subject having the low feeling of visible moisture is recessed inwards.

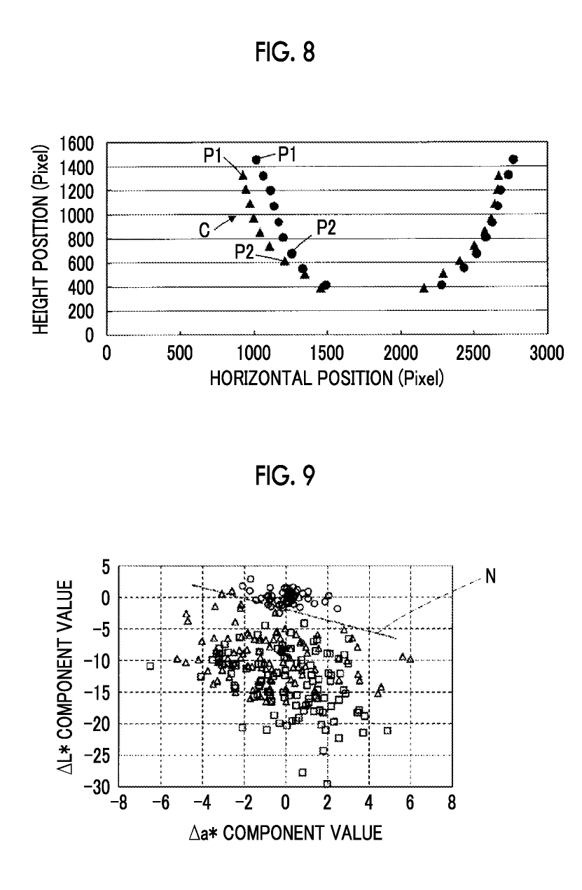

In reality, a state where the contour shape C of the cheek of the subject having the high feeling of visible moisture and the contour shape C of the cheek of the subject having the low feeling of visible moisture are detected and compared with each other is shown in FIG. 8. Here, FIG. 8 shows, when a horizontal axis represents a horizontal position and a vertical axis represents a height position, the contour shapes C of the cheeks by respectively plotting intersections where each horizontal line intersects with the contours, in which the contour shape C of the cheek of the subject having the high feeling of visible moisture is indicated by “▴”, and the contour shape C of the cheek of the subject having the low feeling of visible moisture is indicated by “”. From FIG. 8, it can be understood that the contour shape C of the cheek of the subject having the low feeling of visible moisture is recessed inwards with respect to the contour shape C of the cheek of the subject having the high feeling of visible moisture.

In general, it is known that the contour shape C of the cheek is recessed inwards due to aging, which is a so-called hollow cheek. It may be considered that the recess of the contour shape C of the cheek is a portion that causes an impression of an uneven portion that generates a shadow when generally viewing the face F of the subject, which causes decrease in the feeling of visible moisture.

Thus, the contour recess amount calculation unit 18 calculates a recess amount of the detected contour shape C of the cheek. For example, as shown in FIGS. 7A and 7B, a vertical line S is drawn downward from an intersection P1 in a direction perpendicular to a horizontal line L1, and distances D from the vertical line S to respective intersections are respectively calculated. Further, a total sum of the distances D from the vertical line S to the respective intersections may be set as a recess amount of the contour shape C of the cheek. Here, since a contour shape of the chin below the mouth is greatly affected by bones or the like, it is preferable that the recess amount is calculated with the contour shape C of the cheek being limited in a range from the ear to the mouth so that the recess of the cheek due to aging are reliably reflected.

In this way, the recess of the contour shape C of the cheek shows that the feeling of visible moisture of the face F of the subject becomes higher as the recess amount becomes smaller.

The contour recess amount calculation unit 18 outputs the calculated recess amount of the contour shape C of the cheek to the moisture feeling evaluation unit 5.

In this way, the brightness-color indexes which are respectively calculated in the brightness calculation unit 12, the dullness calculation unit 13, the stain calculation unit 14, and the color irregularity calculation unit 15 of the brightness-color index calculation unit 10 are input to the moisture feeling evaluation unit 5, and the shape indexes which are respectively calculated in the wrinkle calculation unit 16, the pore calculation unit 17, and the contour recess amount calculation unit 18 of the shape index calculation unit 11 are input to the moisture feeling evaluation unit 5.

The moisture feeling evaluation unit 5 makes reference to the reference value database 7 based on the input brightness-color index values and shape index values. In the reference value database 7, a reference value of a feeling of visible moisture obtained by performing functional evaluation in advance is stored with respect to a total index obtained by linearly summing the brightness-color index values and the shape index values using a multiple regression equation or the like. Thus, the moisture feeling evaluation unit 5 calculates the reference value of the feeling of visible moisture depending on the brightness-color index values and the shape index values input from the index calculation unit 4, with reference to the reference value database 7, and evaluates the feeling of visible moisture of the face F of the subject based on the reference value.

The evaluation result of the feeling of visible moisture calculated by the moisture feeling evaluation unit 5 is output and displayed on the display unit 6.

According to this embodiment, since respective physical characteristics of the feeling of visible moisture over the entire face F of the subject is evaluated in a complex manner, it is possible to perform evaluation close to a feeling when generally viewing the face F of the subject, and to evaluate the feeling of visible moisture with high accuracy.

The above-described evaluation of the feeling of visible moisture may be executed by operating a computer configured by input means, a CPU, a memory, and the like by a moisture feeling evaluation program. That is, by operating the computer by the moisture feeling, evaluation program, the image input unit 1 acquires a captured image obtained by imaging a face of a subject, and the CPU executes the preprocessing unit 2, the color space conversion unit 3, the index calculation unit 4, and the moisture feeling evaluation unit 5, to thereby perform evaluation of the feeling of visible moisture with respect to the face of the subject.

Further, in the above-described embodiment, a configuration in which the dullness calculation unit 13 detects dullness portions based on a predetermined threshold value which is set in advance with respect to a ΔL* component, a Δa* component, and a Δb* component, respectively, but the invention is not limited thereto, and any configuration capable of detecting dullness portions may be used.

For example, dullness portions may be detected using a statistic analysis method such as a linear discriminant analysis. FIG. 9 shows results obtained by plotting ΔL* component values with respect to Δa* component values, with respect to non-dullness portions in the reference region R2a, tea dullness portions in the evaluation portion R2, and yellow dullness portions in the evaluation region R2. Here, the non-dullness portions are indicated by “◯”, the tea dullness portions are indicated by “□”, and the yellow dullness portions are indicated by “Δ”, respectively. As a result, it can be understood that it is possible to separate the non-dullness portions, the tea dullness portions, and the yellow dullness portions with an accuracy of about 95% by a discriminant function N. Thus, it can be understood that it is possible to detect dullness portions using the linear discriminant analysis with high accuracy.

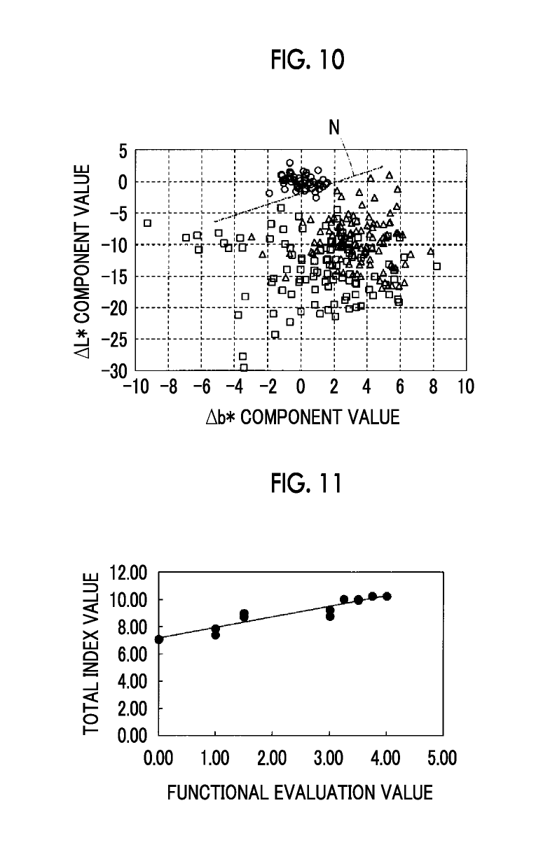

FIG. 10 shows results obtained by plotting ΔL* component values with respect to Δb* component values, with respect to non-dullness portions in the reference region R2a, tea dullness portions in the evaluation portion R2, and yellow dullness portions in the evaluation region R2. Here, the non-dullness portions are indicated by “◯”, the tea dullness portions are indicated by “□”, and the yellow dullness portions are indicated by “Δ”, respectively. Similarly, it can be understood that it is possible to detect dullness portions using the linear discriminant analysis with high accuracy.

Further, in the above-described embodiment, a configuration in which an image captured from the camera C connected to the image input unit 1 is input, but the invention is not limited thereto, and any configuration capable of inputting a captured image may be used.

For example, a captured image may be input to the image input unit 1 through a network from a computer which retains the captured image. The moisture feeling evaluation device evaluates a feeling of visible moisture based on the captured image input from the computer, and stores the evaluation result in a server or the like. Thus, a user is able to browse the evaluation result of the feeling of visible moisture by accessing the server, or to acquire the evaluation result of the feeling of visible moisture through the network from the server.

In reality, an example in which a feeling of visible moisture of a face of a subject is evaluated using the moisture feeling evaluation device will be described.

In this example, a feeling of visible moisture was evaluated using the moisture feeling evaluation device with respect to subjects in their twenties to forties, and functional evaluation of the feeling of visible moisture when three observers generally view the face F of the subject was performed.

FIG. 11 is a graph obtained by plotting a total index value calculated by linearly summing brightness-color indexes and shape indexes obtained using the moisture feeling evaluation device with respect to a functional evaluation value. Here, it is evaluated that, the functional evaluation value is an average value obtained by evaluating the feeling of visible moisture over five stages by the functional evaluation of three observers, in which the value becomes closer to 5, the feeling of visible moisture becomes higher. As a result of calculating a correlation between the total index value and the functional evaluation value based on FIG. 11, a correlation coefficient R² was 0.79.

In this way, by evaluating respective physical characteristics of the feeling of visible moisture over the entire face F of the subject in a complex manner, it can be understood that a correlation with functional evaluation when generally observing the face F of the subject is considerably high, and thus, it is possible to evaluate the feeling of visible moisture with high accuracy.

### EXPLANATION OF REFERENCES

1: image input unit
2: preprocessing unit
3: color space conversion unit
4: index calculation unit
5: moisture feeling evaluation unit
6: display unit
7: reference value database
8: control unit
9: operation unit
10: brightness-color index calculation unit
11: shape index calculation unit
12: brightness calculation unit
13: dullness calculation unit
14: stain calculation unit
15: color irregularity calculation unit
16: wrinkle calculation unit
17: pore calculation unit
18: contour recess amount calculation unit
R1 to R6: evaluation region
R2a: reference region
F: face
C: camera
L1, L2: horizontal line
P1, P2: intersection
C: cheek contour shape
S: vertical line
D: distance from vertical line to intersection
N: discriminant function

What is claimed is:

1. A moisture feeling evaluation device comprising:
an image input unit that receives an input of a captured image obtained by imaging a face of a subject;
a brightness-color index calculation unit that calculates values of brightness-color components including brightness, redness, and yellowness of skin from the captured image input to the image input unit and calculates brightness-color indexes based on the values of the brightness-color components;
a shape index calculation unit that detects an uneven portion of the skin from the captured image input to the image input unit and calculates a shape index based on the amount of the uneven portion; and
a moisture feeling evaluation unit that evaluates a feeling of visible moisture of the face of the subject based on the brightness-color indexes and the shape index.

2. The moisture feeling evaluation device according to claim 1,
wherein the brightness-color index calculation unit includes a brightness calculation unit that calculates the brightness of the skin, a dullness calculation unit that calculates the amount of a dullness portion in the skin, a stain calculation unit that calculates the amount of a stain portion in the skin, and a color irregularity calculation unit that calculates the amount of a color irregularity portion in the skin, and
wherein the shape index calculation unit includes a wrinkle calculation unit that calculates the amount of a wrinkle portion in the skin, a pore calculation unit that calculates the amount of a pore portion in the skin, and a contour recess amount calculation unit that calculates the amount of a recess generated in a cheek contour shape ranging from an ear to the mouth.

3. The moisture feeling evaluation device according to claim 2,
wherein the brightness calculation unit sets a first evaluation region in the captured image, and calculates an average value of brightnesses in the first evaluation region as the brightness of the skin,
wherein the dullness calculation unit sets a second evaluation region in the captured image, detects the dullness portion from the second evaluation region based on a brightness value, a redness value, and a yellowness value, and calculates a total area of the dullness portion, an area ratio of the dullness portion with respect to the second evaluation region, or a shade of the dullness portion, as the amount of the dullness portion,
wherein the stain calculation unit sets a third evaluation region in the captured image, and detects the stain portion where the brightness component value or the color component value is locally changed from the third evaluation region based on its size, and calculates a total area of the stain portion, an area ratio of the stain portion with respect to the third evaluation region, a shade of the stain portion, or the number of the stain portions in the third evaluation region, as the amount of the stain portion, and
wherein the color irregularity calculation unit sets a fourth evaluation region in the captured image, and detects the color irregularity portion where the brightness component value or the color component value is locally changed and its size is larger than that of the stain portion from the fourth evaluation region, and calculates a total area of the color irregularity portion, an area ratio of the color irregularity portion with respect to the fourth evaluation region, a shade of the color irregularity portion, or the number of the color irregularity portions in the fourth evaluation region, as the amount of the color irregularity portion.

4. The moisture feeling evaluation device according to claim 2,
wherein the wrinkle calculation unit sets a fifth evaluation region that extends from the nostrils to the corners of the mouth in the captured image, detects the wrinkle portion where the brightness decreases in the fifth evaluation region, and calculates a total area of the wrinkle portion, an area ratio of the wrinkle portion with respect to the fifth evaluation region, a shade of the wrinkle portion, or the lengths of the wrinkle portion, as the amount of the wrinkle portion,
wherein the pore calculation unit sets a sixth evaluation region in the captured image, detects the pore portion where the brightness component value or the color component value is locally changed and its size is smaller than that of the stain portion from the sixth evaluation region, and calculates a total area of the pore portion, an area ratio of the pore portion with respect to the sixth evaluation region, a shade of the pore portion, or the number of the pore portions in the sixth evaluation region, as the amount of the pore portion, and
wherein the contour recess amount calculation unit detects the cheek contour shape in the captured image, draws a straight line in a downward direction from an outermost portion in the cheek contour shape, and calculates a distance from the straight line to the cheek contour shape as the recess amount.

5. The moisture feeling evaluation device according to claim 1,
wherein the moisture feeling evaluation unit evaluates the feeling of visible moisture based on a linear sum of the brightness-color indexes and the shape index with respect to a reference value of the feeling of visible moisture which is obtained in advance by visually evaluating faces of plural subjects having different brightness-color component values and different amounts of uneven portions.

6. The moisture feeling evaluation device according to claim 5, further comprising:
a database that stores the linear sum of the brightness-color indexes and the shape index with respect to the reference value,
wherein the moisture feeling evaluation unit calculates an evaluation value of the feeling of visible moisture with reference to the database based on the brightness-color index values calculated in the brightness-color index calculation unit and the shape index value calculated in the shape index calculation unit.

7. A moisture feeling evaluation method comprising:
receiving an input of a captured image obtained by imaging a face of a subject;
calculating values of brightness-color components including brightness, redness, and yellowness of skin from the captured image, and calculating brightness-color indexes based on the values of the brightness-color components;
detecting an uneven portion of the skin from the captured image, and calculating a shape index based on the amount of the uneven portion; and
evaluating a feeling of visible moisture of the face of the subject based on the brightness-color indexes and the shape index.

8. A non-transitory computer-readable medium storing moisture feeling evaluation program that causes a computer to execute:
a step of receiving an input of a captured image obtained by imaging a face of a subject;
a step of calculating values of brightness-color components including brightness, redness, and yellowness of skin from the captured image, and calculating brightness-color indexes based on the values of the brightness-color components;
a step of detecting an uneven portion of the skin from the captured image, and calculating a shape index based on the amount of the uneven portion; and
a step of evaluating a feeling of visible moisture of the face of the subject based on the brightness-color indexes and the shape index.
